(19) United States
(12) Patent Application Publication
(10) Pub. No.: US 2011/0214673 A1
(43) Pub. Date: Sep. 8, 2011

(54) Portable Life Support Apparatus Ventilator

(76) Inventor: Edward Masionis, Toronto (CA)

(21) Appl. No.: 12/991,681

(22) PCT Filed: May 1, 2009
(86) PCT No.: PCT/CA2009/000588

§ 371 Date: May 11, 2011

Related U.S. Application Data
(60) Provisional application No. 61/051,620, filed on May 8, 2008.

(51) Int. Cl. A61M 16/20; A61M 16/00

(52) U.S. Cl. 128/205.13; 128/205.24

(57) ABSTRACT

In one aspect, the invention relates to a portable life support device including a ventilator comprising a ventilator bag which combines a pressure relief valve and blow through valve.

### FIELD OF THE INVENTION

The present invention is directed to a portable life support apparatus and particularly to a respiratory support apparatus.

### BACKGROUND OF THE INVENTION

Ventilators for respiratory support require safety and failsafe systems to ensure that the patient has a constant supply of gas for breathing at suitable pressures and tidal volumes. A portable ventilator must implement such safety and failsafe features in space efficient and energy efficient manner. The invention is directed to improvements to ventilator pressure and/or flow control systems

### SUMMARY OF THE INVENTION

In one aspect, the inventions is directed to a ventilator comprising an air pressure generator connected to fresh gas source optionally ambient air and operatively connected to an outer container or can, the can containing and operatively connected to an expandable/contractable inspiratory reservoir, optionally in the form of a bag, the can organized to direct air pressure generated by the air pressure generator to pressurize the outside of the inspiratory reservoir, the inspiratory reservoir optionally adapted to be connected to an inspiratory line and conduit supplying fresh gas to a patient, the can or inspiratory reservoir comprising a blow through valve which set to open (passively or under the control of a controller) at predetermined pressure difference between the can and inspiratory reservoir (optionally when pressure in the can is approximately 10 cm higher than in the bag) in order to direct ambient air into the inspiratory reservoir (when the valve is in the inspiratory reservoir) or inspiratory conduit (when the can is operatively connected to the inspiratory line via the valve) due to a failure to adequately replenish the inspiratory reservoir with the primary source of fresh gas. Preferably the air pressure generator is a blower whose speed and resultant motive pressure on can be controlled by a controller and which can therefore be used exert the required ventilating force as well as PEEP. Optionally, the inspiratory reservoir is a bag. The invention is also directed to an inspiratory reservoir comprising a bifunctional valve which opens, for example, in part, positionally (for example when expansion of the bag exerts tension on a string connected to the valve flap) for example when pressure in the bag expands the bag (for example to a point where pressure in the bag is 2.5 cm higher than in the can) as well when the pressure in the can is higher than in the bag, for example when pressure in the can is 10 cm higher than in the bag, the string serving to concentrate the force exerted on the bag walls when the bag pressure is higher, for example 2.5 cm higher, to open a valve that requires a 10 cm pressure differential if opened by direct pressure on the external side of the flap. The aforementioned ventilator is therefore well adapted be used in a portable or non-portable context to ensure that breathable gas flows to the patient when the primary fresh gas source fails to be supplied.

### BRIEF DESCRIPTION OF THE DRAWINGS

The present invention will now be described by way of example only with reference to the attached drawings, in which:

FIG. 1 is a diagram of one embodiment of a breathing circuit used for conscious or unconscious ventilated patients.

FIG. 2 is a plan view of a one embodiment of the ventilator bag shown in FIGS. 1 and 4.

FIG. 3 is a cross-sectional view of the ventilator bag illustrating detail of the valve shown in FIG. 2.

FIG. 4 is a diagram of the breathing circuit showing an alternate location of the blow-through valve.

### DETAILED DESCRIPTION OF PREFERRED EMBODIMENTS

The following terms are defined as set forth below:

The term “conditioned gas” is used to refer to a gas, optionally conditioned ambient air, having at least one of the following properties: it has a higher content of oxygen than available ambient air, it is less humid than available ambient air, it has a lower nitrogen gas content relative to available ambient air, it comprises exhaled air of a subject that has been scrubbed of carbon dioxide. In a preferred embodiment, the conditioned gas is a gas that has a higher content of oxygen as a result of having been generated by re-breathing circuit and/or an oxygen concentrator and will optionally have been dehumidified and/or scrubbed).

The term “conduit” or “conduit segment” is used broadly to refer to a fluidly intact (pneumatically efficient, and optionally, though not necessarily sealably intact) gas pathway and includes without limitation, tubes and channels of any type that conduct air from place to place.

The term “towards” when used to describe gas flow in a conduit segment (particularly when in operative association with a one way valve) is used to describe unidirectional flow. It will be appreciated that the location of valves including one way valves and points of attachment of conduit segments may often be dictated by convenience or certain advantages which are not necessarily critical to the operation of the structure in which they are incorporated. Accordingly, precise structural linkages may not be material to the operation even if specified in a drawing or descriptions of preferred embodiments of the invention and equivalent arrangements will apparent to persons skilled in art. The term “operative association” and related terms are meant to signify that the precise method of association or location can be variably selected without inventive skill and do not materially affect the operation of some embodiments of the invention. It will also be appreciated that portions of the gas circuit may be left outside the body of the apparatus, particularly disposable, relatively inexpensive, commonly replaceable and technologically trivial parts, and connected by the user via a port designated for such connection, in effect making the port equivalent to those portions of the gas circuit, if added after and secondary to the essential features of the apparatus. Persons skilled in the art of working with respiratory apparatus are attuned to assembly of these types of circuit elements and will readily perceive an assembly of parts as the essential apparatus.

The term “ventilator” includes, without limitation, pressure based ventilators that provide pressure to the airway of the subject to a certain preset level (e.g. 25 cm H2O) or range, and volume based ventilators that control the tidal volume and frequency of the inspiratory flow to the patient. Ventilators of these types could be used for ventilatory assistance of a type that does not require rigorous pressure, volume, frequency controls. A variety of types of ventilatory assistance are known to those skilled in the art including CPAP, BiPAP, pressure controlled, volume controlled, pressure support ventilation, airway pressure release ventilation, inspiratory pause, inspiratory flow profile, proportional assist ventilation, neurally activated ventilatory assistance, assist control ventilation etc. The term “ventilator device” is used broadly to refer to a ventilator and may depending on the context implicitly exclude the gas reservoir component of such a device.

The term “oxygenated” means air having an oxygen content higher than ambient air, optionally having a concentration of at least 40% oxygen.

The term “life support apparatus” (or interchangeably “life support device”) as used herein, generally is used to refer to the apparatus as whole the name contemplating but not implying patient monitoring functions that may or may not be limited to respiratory parameters. However, this term may be used interchangeably with “portable respiratory support apparatus” and “respiratory support apparatus”, among others, in which the primary functions of respiratory support are highlighted in name.

The term “fresh gas” generally means gas entering the patient's breathing circuit that does not contain appreciable amounts of carbon dioxide, and is usually air or oxygen enriched air, although other components may be present as well, such as anesthetic agents or the like.

The term “inspiratory relief valve” means a valve that allows gas, usually ambient air, into a portion of the conduit assembly that is available to the patient to breathe on during an inspiratory cycle in which inspiratory gas, usually in the form of a conditioned gas, is temporarily depleted.

The term “patient airway interface” means a patient interface such as a mask, nasal tube, endotracheal tube, or tracheotomy tube that is fluidly connected to a patient airway.

The term “airway” includes, without limitation, the mouth, trachea, and nose.

The term “processing” with reference to machine intelligence means any handling, merging, sorting or computing of machine readable information using digital or analog circuitry in a way that it is compatible with visual presentation on a screen.

In one aspect, the portable life support system serves to monitor the outcome of respiratory treatment parameters and may also serve to monitor non-treatment parameters of importance to attending medical personnel such as the patient's ECG, heart rate, temperature and blood pressure. Device parameters may also be displayed most notably available battery power and operation modes. Respiratory treatment parameters measured and displayed by the life support system are detailed below. In a general aspect, the portable life support system of the invention contemplates that other forms of treatment and/or monitoring could be provided, measured and/or displayed. The term “treatment” is used broadly to refer to ministrations of any kind, including without limitation provision of respiratory gases, drugs, stimuli, signals etc.

A preferred embodiment of the invention will now be described, and relates to a portable respiratory support apparatus.

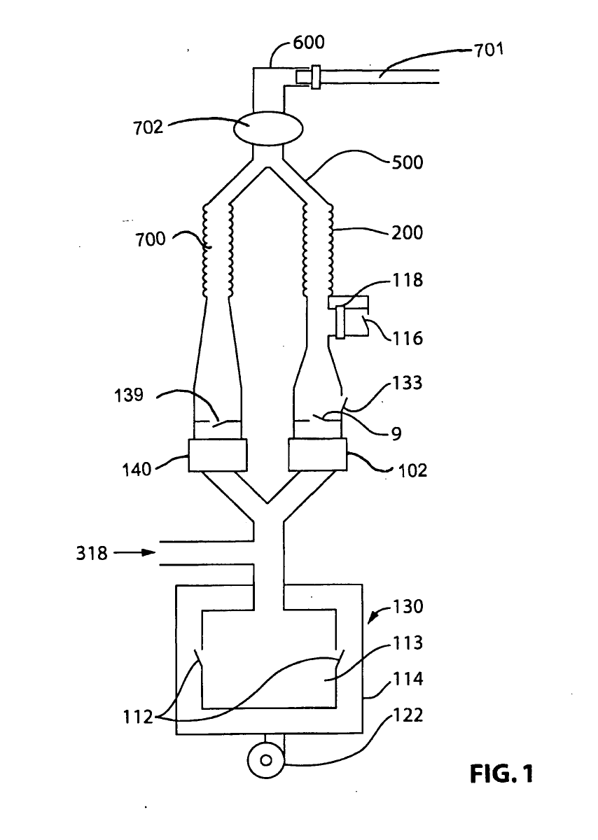

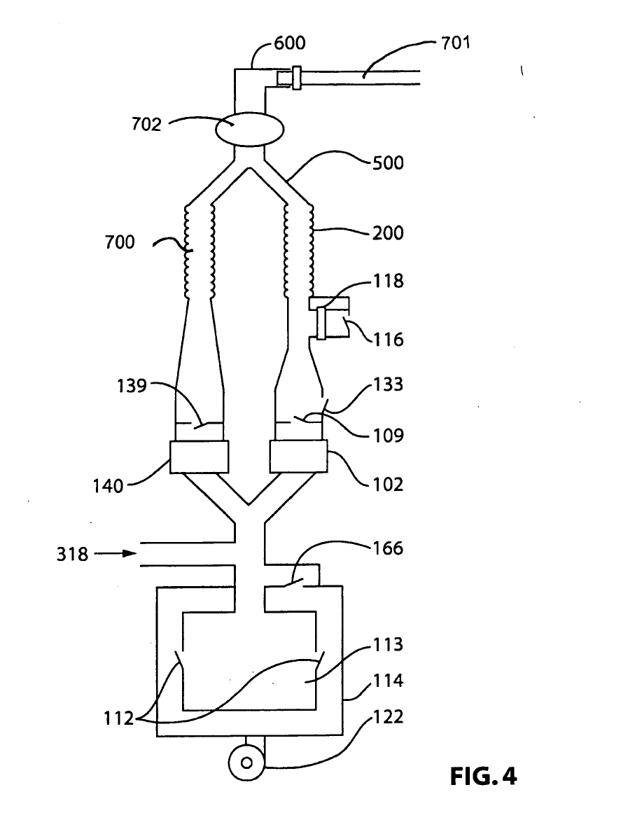

Referring to FIGS. 1 and 4, when fresh gas enters the circuit from the fresh gas inlet port 318 the gas will be directed into a contractible inspiratory reservoir, optionally in the form of the ventilator bag 113. During the inhalation phase the blower 122 draws ambient air through a filter (not shown) into the system and pressurizes the ventilator bag through the instrumentality of an inspiratory reservoir contractor, optionally in the form a pressurized containment chamber or can 114, thus forcing gas accumulated in the ventilator 130 to flow towards the user e.g. patient. When the pressurized gas source, optionally in the form of an air pressure generator, for example a blower 122 pressurizes the ventilator can 114, the gas from the ventilator bag 113 flows through the inspiratory flow sensor 102, through a one-way (1 cm H₂O) inspiratory valve 9 down the (standard 22 mm) inspiratory hose 200, through a Y connector 500 and into the patient interface 600. Pressure in the can 14 may be measured via pressure transducer (not shown) and pressure in the inspiratory line may be measured via airway pressure transducer (not shown).

In FIGS. 1 and 4, other parts identified with common numbers include: endotracheal tube (701), filter (702), extendable expiratory hose (700), expiratory flow sensor (140), expiratory valve (139), and inspiratory flow sensor (102)

In the event, the patient is being ventilated, for example (by synchronized intermittent mandatory ventilation (SIMV), Pressure Support or IMV-Assist Control) and is breathing spontaneously and the patient wishes to inspire a volume of fresh gas that exceeds the volume provided by the system during normal operation, the inspiratory relief valve 116 will open, optionally if the pressure across the valve is less than −6 cm H₂O. When the valve opens ambient air will enter the breathing circuit through the filter 118 and will provide the additional volume desired by the patient. In the event, the aforementioned inspiratory valve 116 does not open, because the valve 116 malfunctions, the ventilator blow through valves 112 will open when the negative pressure on the inspiratory line 115 goes below, for example, −10 cm H2O. When the ventilator blow through valve 112 opens fresh gas from the ventilator can 113 will be directed in the inspiratory line and available for inspiration. Fresh gas will be continuously fed into the ventilator can 114 from environment through the blower 122.

In alternative embodiment shown in FIG. 4, the blow through valve 166 is alternatively positioned and opens from the ventilator can 114 into the inspiratory line instead of the bag 113.

In an alternative embodiment, the inspiratory reservoir 113 could comprise some other suitable vessel, such as a bellows, instead of a bag. In summary, noteworthy operational and safety features:
(i) Running the blower 122 during the patient's exhalation the system can provide an adjustable PEEP between 0-10 cm H₂O.
(ii) If an inspiratory valve does not open, because the valve malfunctions, the ventilator blow through valve 112 will open when the negative pressure on the inspiratory line goes below −10 cm H2O. When the ventilator blow-through valve 112 opens fresh gas from the ventilator can 114 originating from the blower 122 will be available to the patient via the inspiratory line.

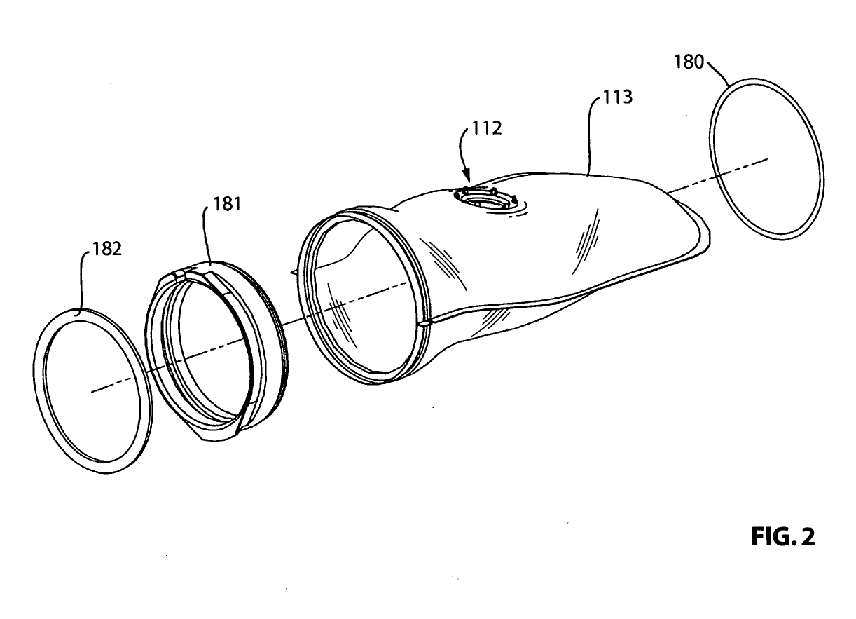

As shown in FIG. 2, in one embodiment bag 113, optionally made of urethane, ring 180 may be used to seal the bag to a connector ring 181 which connects to the bag 113 to the valve block (not shown). Alternatively, ring 180 may be replaced by glue of use of RF/sonic welding. Sealing ring 182 enables a tight seal to the valve block.

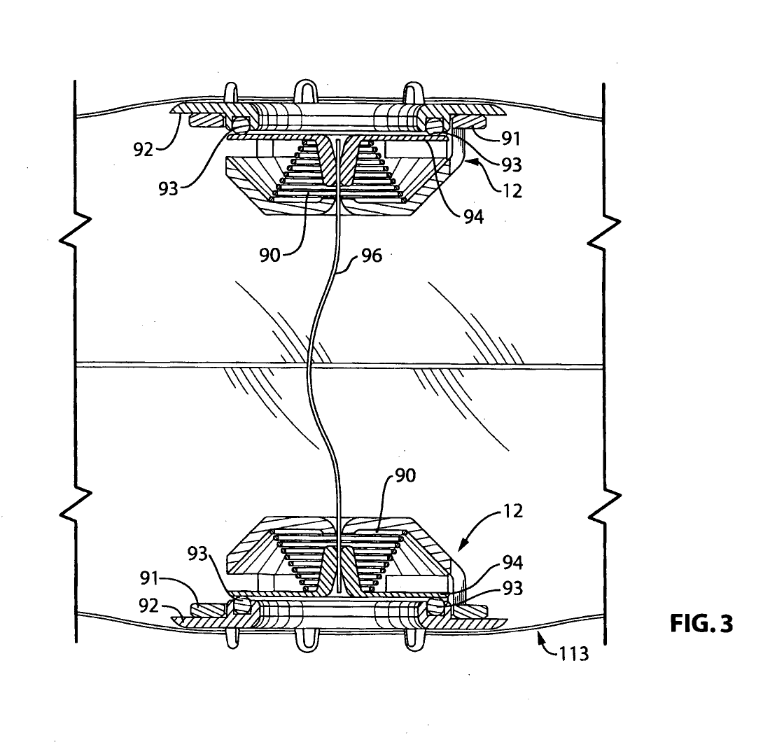

FIG. 3 shows a cross-section through the ventilator bag 113 showing how a force translator, optionally in the form of positional translator, for example string 96 (which serves as one example of an actuator for opening the multifunction valve) interconnects valve flaps 94 which are biased in closed position by biasing means, optionally in the form of springs 90 which are held in position by cage 91 which in turn abuts against flange 92. Flange 92 is sealingly attached to the bag when the bag expands to a size which causes the string 96 to pull on valve flaps 94, a −2.5 cm pressure differential relative to the can concentrates the force against the bag over a smaller surface area (the flaps) and is sufficient to open a valve biased closed by spring 90 which is which geared to open in the event that the pressure outside the bag is, for example, 10 cm greater than the pressure in the bag (blow through valve). In this manner the valves 112 can operate as both blow-through and expiratory relief valves. Sealing ring 93 is also shown in FIG. 3.

The system may be fitted with a safety pressure relief valve or pop-off 133 that has, for example, an opening pressure approximately equal to the maximum desired airway pressure, for example, 60 cm H₂O. Optional ranges for ventilation parameters include:

1. Inspired O₂ concentrations of 21% to 93%—For increased ease of use, 3 presets may be settable by the user of 21%, 40%, and 85%. Tidal volumes may be settable between 400 ml and 1 litre (e.g in increments of 100 ml), which are useful for adult ventilation.

2. Breath Frequency: between 8 and 20 per minute

3. PEEP: 0-25 cm H₂O optionally with settings incremented in 5 cm H₂O

4. Inspiratory: Expiratory ratio between 1:1 AND 1:2—this is typically adjusted automatically based on tidal volume, breath frequency, and blower flow rate.

5. End Inspiratory or end expiratory Pause with pressure hold.

If the system reaches the maximum airway pressure limit set on the ventilator control, the blower may stop blowing and may switch into a constant PEEP mode.

In spontaneous breathing mode, it is helpful for ease of use to provide a concentration of 40% O₂, since most adults require less than 8 LPM of FGF, and providing this concentration requires a oxygen source capable of producing 2.2 LPM of 90% O2 which can be made relatively small (<10 lbs.).

The patient can breathe at any frequency and with any tidal volume in spontaneous mode.

1. A life support apparatus, including a ventilator, the ventilator comprising:
(a) a contractible inspiratory reservoir which is adapted to be contracted to expel a reservoir gas responsive to a source of fluid pressure, the contractible inspiratory reservoir including an aperture for establishing a fluidly efficient connection to an inspiratory conduit connected to a user interface;
(b) an inspiratory reservoir contractor operatively associated with at least a portion of the exterior surface of the contractible inspiratory reservoir for exerting a compressive force on the inspiratory reservoir, the inspiratory reservoir contractor including a port fluidly connected to a source of breathable gas through which a breathable gas (i.e. safe for breathing) under pressure is introduced against the exterior surface of the inspiratory gas reservoir to compress the inspiratory reservoir;
(c) a valve located in the contractible inspiratory reservoir or inspiratory reservoir contractor through which the breathable gas under pressure is adapted to be channeled into the inspiratory conduit for inspiration by the user in the event that the user is unable to get sufficient gas for inspiration in the course of operation of the ventilator.

2. The life support apparatus of claim 1, wherein the inspiratory reservoir contractor comprises a containment chamber for housing the flexible ventilator bag.

3. The life support apparatus of claim 2, and wherein the source of pressurized gas is a blower and wherein the containment chamber is operatively connected to the blower and wherein air blown into the containment chamber exerts pressure on the contractible inspiratory reservoir to compress it.

4. The life support apparatus of claim 3, comprising an ambient air inlet in fluid communication with said blower and wherein the contractible inspiratory reservoir contains at least one valve (e.g. a blow through valve) that is adapted to open at a pressure differential between the containment chamber and the interior of the contractible inspiratory reservoir that is indicative of a failure of the contractible inspiratory reservoir to supply the gas demands of the user and wherein the blower propels ambient air entering the ambient air inlet into the containment chamber to compress the contractible inspiratory reservoir.

5. The life support apparatus according to claim 4, wherein the pressure is higher differential is 5-15 cm H₂O higher in the containment chamber.

6. The life support apparatus according to claim 5, wherein the pressure differential is 10 cm H₂O higher in the containment chamber.

7. The life support apparatus according to any of the preceding claims, wherein the contractible inspiratory reservoir comprises a pressure relief valve that opens to reduce pressure in the contractible inspiratory reservoir when the pressure inside the contractible inspiratory reservoir exceeds that of the containment chamber.

8. The life support apparatus according to claim 7, wherein the pressure relief valve and the blow through valve are combined into a single multifunction (i.e. two or more functions) valve.

9. The life support apparatus according to claim 8, wherein the multifunction valve opens under a first condition of differential pressure in which the pressure in the containment chamber is higher and a second condition of differential pressure in which the pressure inside the bag is higher an wherein the first and second conditions of different are of a different magnitude, preferably by organizing the pressure on one side the bag to be applied to a smaller surface area (e.g. the valve flap) relative to the other side of the bag (e.g a substantial part of the bag wall) and translating the force generated by the pressure applied to the larger surface area into a counterforce needed to open the valve flap against the biasing means.

10. The life support apparatus according to claim 9, wherein the multifunction valve opens at a first condition of differential pressure that is 2 to 6 fold greater than the second condition of differential pressure.

11. The life support apparatus according to claim 9, wherein the contractible inspiratory reservoir is a ventilator bag and the multifunction valve opens at first condition of differential pressure that is 3 to 5, optionally 4 fold greater than the second condition of differential pressure.

12. The life support apparatus according to claim 11, wherein the multifunction valve opens at first condition of differential pressure that is 10 cm H₂O and a second condition of differential pressure that 2.5 cm H₂O.

13. The life support apparatus according to claim 9, wherein the multifunction valve comprises a valve flap, a valve seat, biasing means for biasing the valve flap in a closed position against the valve seat and a force translator for directly or indirectly translating the force applied to the walls of the ventilator bag as a result of the first or second condition of differential pressure in a direction which substantially opposes or enhances that of the biasing means.

14. The life support apparatus according to claim 13, wherein the force translator is a positional translator that translate the movement of the interior walls of the bag (generated as a result of the second condition of differential pressure) on the valve flap or an actuator operatively associated with the valve flap in a manner which opposes the position of valve flap imposed by the biasing means.

15. The life support apparatus according to claim 14, wherein the positional translator is a string of pre-defined length.

16. The life support apparatus according to claim 9 or 14, comprising two multifunction valves on opposite walls of the bag and a force translator that simultaneously translates the forces exerted by the second condition of pressure on opposing walls of the bag onto both valve flaps in a direction which opposes the direction of the force imposed each of biasing means.

17. The life support apparatus according to claim 16, wherein the force translator is a positional translator.

18. A ventilator as characterized in any of claims 1 to 17, a ventilator bag as characterized in any of claims 4 to 17 and a multifunction valve or valve set as characterized in any claims 8 to 17.
